(19) United States
(12) Patent Application Publication
(10) Pub. No.: US 2015/0038681 A1
(43) Pub. Date: Feb. 5, 2015

(54) METHOD OF PREPARING A PRODUCT BY DIALYSIS

(71) Applicant: TETEC Tissue Engineering Technologies AG, Reutlingen (DE)

(72) Inventors: Juergen Mollenhauer, Reutlingen (DE); Heinz W. Joseph, Freiburg (DE); Helmut Wurst, Kusterdingen (DE)

(21) Appl. No.: 14/450,543

(22) Filed: Aug. 4, 2014

(30) Foreign Application Priority Data
Aug. 5, 2013 (DE) 10 2013 215 380.5
Sep. 11, 2013 (DE) 10 2013 218 239.2

(51) Int. Cl. C07K 1/107; C07K 14/765

(52) U.S. Cl. CPC C07K 1/1077; C07K 14/765
USPC 530/363

(57) ABSTRACT

A method of preparing a product, preferably in the form of a functionalized high molecular weight component such as, for example, a functionalized protein includes passing a first liquid comprising a component A along one side of a semi-permeable membrane and passing a second liquid comprising a component B along another, preferably opposing, side of the membrane, wherein the membrane excludes passage of component A but allows passage of component B, and after passage of component B through the membrane, a chemical reaction takes place between component A and component B, preferably with the formation of covalent bonds.

### TECHNICAL FIELD

This disclosure relates to a method of preparing a product, preferably in the form of a functionalized high molecular weight component, by dialysis, to a product which is prepared or may be prepared by such a method and also to the use of a dialysis apparatus to perform such a method.

### BACKGROUND

Preparation of medicaments or medicinally effective compositions such as injectable compositions described in WO 2009/095223 A1, must generally be conducted in GMP clean rooms (Good Manufacturing Practice clean rooms) for reasons of quality assurance of the manufacturing processes and manufacturing environment.

Medicaments are very frequently prepared by open process technologies in which individual method steps such as preparation, purification and optionally reduction of sample volumes, proceed compartmentalized or spatially separate from one another.

Such process technologies, however, render control of sterility and also stabilization of an aseptic production process more difficult to a considerable degree. Performing sterile or aseptic production processes is also rendered more difficult by the long production times which typically result from open process procedures.

Overall, the methods of preparing products to GMP quality described above require a considerably increased qualification and validation effort.

A method of removing unwanted or potentially harmful protein bound substances (PBS) from a protein-containing liquid such as plasma or blood by dialysis is known from DE 693 30 179 T2.

It could therefore be helpful to provide a preparation process for products for which particularly the achievement of GMP quality is required and disadvantages of prior methods are at least largely avoided.

### SUMMARY

We provide a method of preparing a product including passing a first liquid comprising component A along one side of a semi-permeable membrane and passing a second liquid comprising component B along another side of the membrane, wherein the membrane excludes passage of component A but allows passage of component B and, after passage of component B through the membrane, a chemical reaction takes place between component A and component B.

We also provide a product, prepared or which may be prepared by the method according to claim 1.

### BRIEF DESCRIPTION OF THE DRAWING

FIG. 1 schematically shows the design of a dialysis device 100 that performs our methods.

### DETAILED DESCRIPTION

Our method is a method of preparing a product, particularly with subsequent purification and/or concentration, preferably a product which complies with GMP standards.

The product is preferably a high molecular weight and preferably functionalized component such as a functionalized protein, for example.

The method comprises the following steps:

passing a first liquid comprising a component A along one side of a semi-permeable membrane, and

passing a second liquid comprising a component B along another, preferably opposing, side of the membrane.

The membrane excludes passage of component A. However, the membrane allows passage of component B.

After passage of component B through the membrane, a chemical reaction takes place between component A and component B, preferably with the formation of covalent bonds.

In other words, therefore, we provide a chemical preparation process for products, based on dialysis, i.e. substance transport through a semi-permeable membrane.

The reaction which takes place between the components A and B may lead to the desired product.

However, it may be distinctly preferable that the reaction to prepare the product requires a further component, which is explained in more detail below.

The expression “semi-permeable membrane” means a membrane which is “semi-permeable” or “partially permeable” and—as stated above—is impermeable to component A, while being permeable (at least) to component B.

The expression “functionalized” means any completed process by which the product, by addition of groups of atoms or functional groups, for example, is given a function which the product does not normally possess.

Preferably, the method is a method that functionalizes, particularly derivatizes a product, in particular a high molecular weight component.

The expression “high molecular weight component” means a component of which the molecular weight is greater than the molecular weight cut-off (MWCO) of the semi-permeable membrane.

Preparation of the product preferably includes—as already mentioned—purification and/or concentration thereof.

The second liquid is preferably guided along the membrane countercurrent to the first liquid (or vice versa). Application of the countercurrent principle enables, with particular advantage, concentration of the product prepared and in addition a reduction of the amount of liquid required for this purpose, for example, the amount of dialysis buffer. A further advantage of the countercurrent principle is the precise adjustment of the osmotic pressure. Moreover, the effectiveness of the mass transfer across the membrane increases by the countercurrent principle.

The first liquid and the second liquid are in appropriate examples both guided by pumps, preferably peristaltic pumps, along the membrane. The use of peristaltic pumps enables generation of opposing flows of the first liquid and the second liquid in a particularly advantageous way.

To control the reaction between the components A and B, it may be preferable to vary the concentration of component A in the first liquid and/or the concentration of component B in the second liquid, particularly to vary the concentrations gradually (gradient mode).

For example, to accelerate the reaction which takes place between the components A and B, the concentration of component A in the first liquid and/or the concentration of component B in the second liquid may be increased, particularly increased gradually (gradient mode).

The first liquid and the second liquid are each preferably guided along the membrane via a closed loop. Accordingly, the first liquid may be guided along the membrane via a first closed loop and the second liquid via a second closed loop. The disadvantages discussed above, particularly in connection with open methods can thereby be avoided with respect to sterility control and stabilization of an aseptic production process.

To achieve a pressure gradient across the membrane, it can be provided in a further configuration that the first liquid, particularly in the first loop mentioned in the previous paragraph, is circulated in suction mode and the second liquid, particularly in the second loop mentioned in the previous paragraph, is guided in pressure mode.

Particularly advantageously, preparation, in particular functionalization, and also subsequent purification and/or concentration of the product is carried out via the closed circulation routing described in the previous paragraph.

The membrane is typically a constituent part of a membrane reactor. A dialyser is preferably used as the membrane reactor. In other words, the preparation of the product is preferably carried out in a dialyser which contains the semi-permeable membrane.

The dialyser can be, in particular, a capillary dialyser. A capillary dialyser is typically composed of a casing in which a large number, for example, up to 10 000 of hollow fibres are arranged parallel to one another or largely parallel to one another. The hollow fibres may have a length of 15 cm to 30 cm, in particular 20 cm to 25 cm. Furthermore, the hollow fibres may have a diameter of 200 μm to 400 μm, in particular of ca. 300 μm. The hollow fibres may also have a wall thickness of 30 μm to 50 μm, in particular of ca. 40 μm.

When using a capillary dialyser, preparation of the product is preferably carried out such that the first liquid containing component A is guided inside the hollow fibres and the second liquid containing component B is guided outside the hollow fibres, preferably in countercurrent. By using a capillary dialyser, a rapid and particularly efficient preparation of the product is possible due to the large specific membrane surface area resulting from the large number of hollow fibres.

The dialyser described in the examples above may be present in particular in the form of a dialysis cassette.

Preparation of the product is particularly preferably carried out by a dialysis apparatus or equipment. The use of dialysis equipment, which is typically used for haemodialysis, has the great advantage that it is equipment already qualified or validated in the field of medical technology, and it is possible therefore to refer back to qualification or validation documentation already in place for the equipment. In addition, the use of dialysis equipment enables a partial automation of the production process.

In terms of suitable dialysis equipment, there are in principle no limits.

However, suitable dialysis equipment typically has a dialyser as a central feature, in which substance exchange may take place via a semi-permeable membrane in the manner described above (passage of component B but not of component A) between the first and second liquid. Preferably, the first liquid containing component A is guided along the membrane via a loop system referred to as extracorporeal blood circulation and the second liquid containing component B via a loop system referred to as dialysate circulation. The structure of dialysis equipment is sufficiently known that further comments are needed.

Overall, therefore, dialysis equipment fulfills all system requirements to perform the method.

In contrast to manual dialysis, the use of dialysis equipment has the further advantage that the liquid requirement, particularly with respect to the first liquid, can be reduced.

The membrane used in the method preferably has a molecular weight cut-off (MWCO) of 2 kDa (kilodalton) to 20 kDa (kilodalton), in particular 3 kDa (kilodalton) to 18 kDa (kilodalton), preferably 5 kDa (kilodalton) to 15 kDa (kilodalton).

Furthermore, the membrane can be prepared from materials selected from the group comprising ceramics, graphite, metals, metal oxides, polymers, particularly organic polymers, and mixtures thereof.

For example, the membrane may be prepared from a polymer selected from the group comprising polysulfones, polyamides, polycarbonates, polyesters, acrylonitrile polymers, polyvinyl alcohols, acrylate polymers, methacrylate polymers, cellulose acetate polymers and mixtures thereof.

Membranes which may be used are known as such and are described, for example, in Kirk-Othmer, Encyclopedia of Chemical Technology, 3rd edition, volume 7 (1979), 564-579, particularly 574-575, volume 12 (1980), 492-517 and volume 15 (1981), 92-131. In addition, suitable membranes are also described in Ullmann's Encyclopedia of Industrial Chemistry, 5th Edition, Volume A 16 (1990), 187-263.

The second liquid may, in addition to component B, comprise a further component C. Alternatively, after the second liquid containing component B, a third liquid can be guided along the membrane which comprises a further component C. In the example described in this paragraph, the membrane is preferably also permeable to component C.

After passage through the membrane of the optional component C additionally provided, a chemical reaction may also take place between component A and component C, preferably with the formation of covalent bonds.

Component B and the optional component C additionally provided preferably react at different positions to component A. In particular, components B and C react with different atom groups, preferably functional groups, to component A.

Component B and/or optional component C additionally provided is preferably a component having a molecular weight of <5 kDa, particularly <3 kDa, preferably <2 kDa.

Component B and/or optional component C additionally provided is preferably a thiol reactive compound, i.e. a compound capable of reacting with thiol and/or thiolate groups of component A. The reaction preferably proceeds selectively and may in particular be based on a Michael addition.

Suitable thiol reactive compounds may be selected from the group comprising maleimide compounds, vinyl sulphone compounds, acrylate compounds, alkyl halide compounds, azirine compounds, pyridine compounds, thionitrobenzoic acid compounds, aryl compounds, derivatives thereof and mixtures thereof.

Component B and/or optional component C additionally provided is preferably a maleimide compound or a maleimide derivative.

Component B is particularly preferably 3-maleimidopropionic acid (or N-maleoyl-β-alanine).

As an alternative to or in combination with the previous examples, component B and/or optional component C additionally provided is an amino group-reactive compound. An amino group-reactive compound means a compound capable of reacting with amino groups, preferably primary amino groups, of component A.

Component B and/or optional component C additionally provided is preferably an amino group-reactive and thiol-reactive compound.

Component B and/or optional component C additionally provided is particularly an active ester. An active ester means an ester having an activated acyl group, i.e. an ester with elevated acylation potential. The activation ability preferably depends on the presence of a good leaving group covalently bound to the acyl carbon atom.

Component B and/or optional component C additionally provided is a maleimide-functionalized active ester, i.e. an active ester having a maleimide unit.

Component B and/or optional component C additionally provided is preferably 3-maleimido-propionic acid N-hydroxysuccinimide ester.

The liquids may be selected from the group comprising solutions, dispersions and suspensions. Water and/or dimethylformamide are suitable as solvent, dispersion medium or suspension medium.

Particular preference is given to the use of a solution containing component A as first liquid, a solution containing component B as second liquid and optionally a solution containing component C as an optional third liquid additionally provided.

Component A in the first liquid may be present at a concentration of 100 μmol/l to 1.5 mmol/l, particularly 200 μmol/l to 1 mmol/l, preferably 300 μmol/l to 800 μmol/l.

Component B in the second liquid may be present at a concentration of 35 mmol/l to 530 mmol/l, particularly 70 mmol/l to 350 mmol/l, preferably 105 mmol/l to 280 mmol/l.

Optional component C provided in addition in the second liquid or in a third liquid may also be present at a concentration of 35 mmol/l to 530 mmol/l, particularly 70 mmol/l to 350 mmol/l, preferably 105 mmol/l to 280 mmol/l.

Components A, B and/or optional component C provided may be guided along the membrane at a flow rate of 25 ml/min to 500 ml/min, particularly 100 ml/min to 400 ml/min, preferably 200 ml/min to 300 ml/min, per square metre of the membrane surface.

Advantageously, the method may be carried out at room or ambient temperature, particularly at 20° C. to 30° C.

Furthermore, the method may be carried out over a time period of 15 min to 5 h, particularly 30 min to 3 h, preferably 45 min to 2 h.

Component A is preferably a high molecular weight component. For example, component A may have a molecular weight of 10 kDa to 90 kDa, particularly 15 kDa to 80 kDa, preferably 20 kDa to 70 kDa.

In addition, component A may be a synthetic or biological polymer, particularly a naturally occurring or recombinantly produced polymer.

Component A may be of human or xenogenic origin, particularly bovine, porcine or equine origin.

For example, component A may be selected from the group comprising proteins, particularly serum proteins such as, for example, albumin, enzymes, antibodies, extracellular proteins such as, for example, collagen, elastin, reticulin, fibronectin or the like, hormones, growth factors, cytokines, polysaccharides, particularly mucopolysaccharides such as, for example, hyaluronic acid, salts thereof, derivatives thereof, conjugates thereof and mixtures thereof.

Particularly preferably, component A is albumin. The albumin preferably has a molecular weight of 66 kDa to 67 kDa, particularly 66 kDa.

Component A is preferably serum albumin, particularly bovine or human serum albumin.

The product to be prepared may be selected in principle from the group comprising medicaments, pharmaceutical compounds, medicinal products, food and cosmetic products.

The product to be prepared is preferably selected from the group comprising functionalized proteins such as, for example, functionalized albumin, functionalized enzymes, functionalized antibodies, functionalized extracellular proteins, functionalized hormones, functionalized growth factors, functionalized cytokines, functionalized polysaccharides, functionalized mucopolysaccharides, salts thereof and mixtures thereof.

The product to be prepared is preferably a maleimide-functionalized protein, particularly maleimide-functionalized albumin.

The product to be prepared is particularly preferably the hydrophilic polymer described in WO 2009/095223 A1, particularly functionalized or modified albumin, for which reason the subject matter of that PCT patent application is incorporated by reference.

The first, second and/or optional third liquid provided is present in relevant examples in each case as aqueous buffer solution, in particular aqueous phosphate buffer solution, and/or aqueous electrolyte solution.

In particular, salts may be provided in the first, second and/or optional third liquid provided which are selected from the group comprising sodium chloride, potassium chloride, sodium dihydrogen phosphate, disodium hydrogen phosphate, sodium phosphate, potassium dihydrogen phosphate, dipotassium hydrogen phosphate, potassium phosphate, magnesium chloride, calcium chloride, sodium lactate, glucose monohydrate and mixtures thereof.

We also provide a product, preferably in the form of a functionalized high molecular weight component such as, for example, a functionalized protein, which is prepared or may be prepared according to one of the methods described above. With regard to further features and advantages of the product, particularly the method of preparation thereof, reference is made in full to this disclosure to avoid unnecessary repetitions.

We further provide the use of a dialyser or a dialysis cassette to perform the method. With regard to further features and advantages of the dialyser or the dialysis cassette and also the method, reference is likewise made in full to this disclosure.

Finally, we provide the use of a dialysis device or a dialysis apparatus or equipment that performs the method. With regard to further features and advantages of the dialysis device or the dialysis apparatus or equipment and also the method, reference is also made in full to this disclosure.

At this point, advantages shall be summarized once more as follows:

The method, with particular advantage, allows a completely closed process procedure, whereby a safe aseptic product preparation is possible.

The method also allows purification and/or concentration of the product prepared.

In particular, it is possible to carry out the preparation of the product, including a subsequent purification and/or concentration of the same, in a closed system.

It is particularly advantageous that the method can be carried out using a dialysis apparatus or equipment. Dialysis apparatus or equipment are sufficiently proven in the medicinal field such that extensive qualification and validation documentation are available, which guarantee a successful process procedure.

Also, the use of mobile dialysis apparatus can contribute to a further simplification and, in particular, flexibility of the method.

A further advantage is that the method constitutes a mild process with regard to the product to be prepared, which, in particular, may proceed at room temperature.

Also advantageous are the (distinctly) shorter preparation times, which additionally increases the aseptic safety standard.

Additionally advantageous is that the method allows working with medium scale batches. At present, batches can in fact only be run on a laboratory scale or in large-scale plants.

A further advantage to highlight is that the method requires distinctly lower volumes of dialysis buffer solutions than generic methods.

Finally, the method is advantageous in that it allows the operating process to be automated and, in particular, to be scaled.

Further features and advantages are apparent from the following description of a preferred working example. It will be appreciated that the features mentioned above, and those still to be illustrated below, may be applied not only in the respective combinations indicated, but also in other combinations or in isolation, without leaving the scope of this disclosure,

The device 100 comprises a membrane reactor 110 and two reservoirs 140 and 150 as shown in the Drawing.

The membrane reactor 110 comprises a semi-permeable membrane. The membrane is impermeable to a component A, for example, while it is permeable to a component B. The membrane reactor 110 is generally a dialyser or a dialysis cassette.

The reservoir 140 is provided for storage of a first liquid containing a component A. Component A can be a protein, particularly albumin.

The container 150 is provided for storage of a second liquid containing a component B. Component B can be an amino group-reactive and thiol-reactive compound such as for example 3-maleimidopropionic acid N-hydroxysuccinimide ester.

The reservoir 140 has an inlet 142 and an outlet 148.

Accordingly, the container 150 has an inlet 152 and an outlet 158.

The outlet 148 connects via a feed line 120 to an inlet 112 of the membrane reactor 110, while the inlet 142 connects via a removal line 130 to an outlet 118 of the membrane reactor 110.

Correspondingly, the outlet 158 connects via a feed line 120′ to an inlet 112′ of the membrane reactor 110 and the inlet 152 connects via a removal line 130′ to an outlet 118′ of the membrane reactor 110.

A pump 135 or 135′ is preferably arranged between the outlet 118 and the inlet 142 on one side and the outlet 158 and the inlet 112′ on the other side. The pump can be, for example, a peristaltic pump.

The membrane reactor 110, together with lines 120 and 130 and the reservoir 140, forms a first closed loop, and together with the lines 120′ and 130′ and the container 150 a second closed loop.

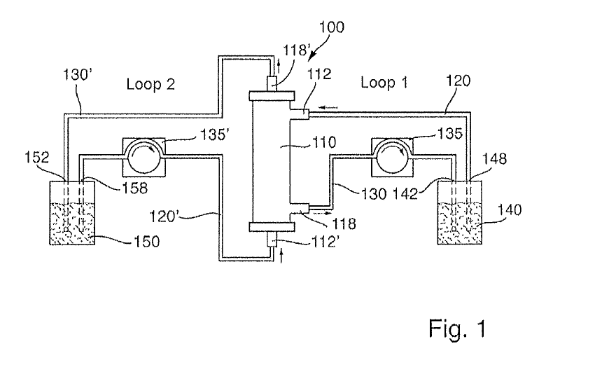

There follows an exemplary illustration of the method with reference to the device shown in FIG. 1:

The first liquid containing component A, stored in the container 140, is fed via the feed line 120 to the membrane reactor 110 and within the membrane reactor 110 is guided along the semi-permeable membrane thereof. The first liquid is again led away from the membrane reactor 110 via the removal line 130 and fed back into the reservoir 140. The first liquid can be continuously conducted around the first loop by the pump 135.

Correspondingly, the second liquid containing component B, stored in the container 150, is fed via the feed line 120′ to the membrane reactor 110 and within the membrane reactor 110 is guided along the semi-permeable membrane thereof. The second liquid is again led away from the membrane reactor 110 via the removal line 130′ and fed back into the container 150. The second liquid can be continuously conducted around the second loop by the pump 135′.

During the passing of the second liquid along the semi-permeable membrane contained in the membrane reactor 110, some of the amount of component B being guided along passes through the membrane and reacts on the opposing side of the membrane with component A being guided along on that side. The reaction between the components A and B preferably depends on formation of covalent bonds.

As mentioned above, when component A is a protein, albumin, for example, and when component B is 3-maleimidopropionic acid N-hydroxysuccinimide ester, then maleimidopropionic acid N-hydroxysuccinimide ester molecules preferably react after passage through the semi-permeable membrane with primary amino groups of the protein, which forms maleimide-functionalized protein.

### WORKING EXAMPLE

### 1. Materials and Methods

1.1 Solutions

Albumin solution: 10 mg/ml of human serum albumin (fraction V, Calbiochem, Cat. No. 12668) in 200 ml of 0.2 mol/l Na₂HPO₄, 0.4 mol/l boric acid, adjusted to pH 8.1 with NaOH.

SMP solution 1: 2000 mg of 3-maleimidopropionic acid N-hydroxysuccinimide ester (Obiter Research, Champaign Ill., USA, Cat. No. OBT-104) dissolved in 25 ml of N,N-dimethylformamide (Sigma-Aldrich, Cat. No. 22, 705-6).

SMP solution 2: 250 ml of 50 mmol/l Na citrate (pH 3.6), 10% (v/v) SMP solution 1.
Sodium acetate: 0.3 M NaOOCCH₃ (pH 4.7)
Sodium dihydrogen phosphate: 10 mmol/l NaH₂PO₄

1.2 Equipment
Dialysis cassette: H.E.L.P. Ultrafilter SMC 1.8
Tubing: Pharmed® NSF-51

Dialysis cassette, tubing and tubing connectors were provided by P. Mandry (MAT Adsorption Technologies GmbH & Co. KG, Obernburg).

Gel chromatography investigations were carried out using a Smart Chromatography System (Pharmacia) and a Superose 6 10/300 GL column (GE Healthcare). UV spectroscopy was carried out using a Spectronic Genesys 2 instrument.

1.3 Gel Formation

10×CB (pH 7.2) and PEG-Link (both from Cellendes GmbH, Reutlingen) were used as buffer and crosslinker respectively for testing the gel formation of the maleimide-functionalized albumin.

### 2. Procedure

The functionalization was carried out in a membrane reactor, which is a constituent part of a system, as shown in FIG. 1, at 4° C.

To functionalize albumin with maleimide groups, 200 ml of albumin solution were circulated in the first loop (see FIG. 1) at a flow rate of 35 ml/min. In the second loop (see FIG. 1), 52 ml of SMP solution 2 were counter-circulated at a flow rate of 100 ml/min for 30 min.

Since the albumin solution was circulated in the first loop in suction mode through the dialysis cassette and the SMP solution 2 was circulated in the second loop in pressure mode, a pressure gradient formed across the dialysis membrane. On account of this pressure drop, the reservoir for the SMP solution 2 was completely emptied after 30 min.

Following functionalization, the empty reservoir for the SMP solution 2 was replaced by a container of one liter of sodium acetate solution. This solution was not circulated but was pumped through the membrane reactor at a flow rate of 100 ml/min and collected in a separate vessel.

Since the volume of the albumin solution had increased after acidification to ca. 600 ml, configuration of the membrane reactor was changed to enable concentration of the albumin solution. The pumping direction of both peristaltic pumps was reversed, which also reversed the pressure gradient across the membrane. This pressure gradient was further increased by an adjustable hose clip, which was inserted in the first loop, to generate a back pressure in the membrane reactor. The liquid passing from loop 1 into loop 2 by the pressure gradients was collected at a pumping rate of 20 ml/min. The reactor was operated in this mode for 40 min, until the volume of the albumin solution in the storage vessel of loop 2 had been reduced to 200 ml.

The hose clip in loop 1 was again removed and the sodium dihydrogen phosphate solution was pumped through loop 2 at a flow rate of 20 ml/min to purify the albumin solution. The eluate from loop 2 was collected separately. After passing through 1.8 liters of purification solution, a sample for analysis was taken from the albumin loop and the purification continued until a total of 3 liters of sodium dihydrogen phosphate had been pumped through the reactor.

The concentration of the albumin solution after purification was effected in the same configuration as in the first concentration. The reactor was operated in this mode until the storage vessel of the albumin solution had been completely emptied. Subsequently, the reactor was emptied, wherein 90 ml of albumin solution were obtained.

To determine the purity, the original albumin and the material following the various process steps were both investigated by gel filtration chromatography to detect contamination with low molecular weight substances. The albumin used originally had only low levels of contamination with low molecular weight substances. After processing with the SMP solution 2, in contrast, a strong absorption signal in the low molecular weight range was measured, which was generated by maleimide-NHS groups and the citrate from the SMP solution. After purification with 1.8 liters of sodium dihydrogen phosphate, this signal was already considerably weaker and in the end product the signal from low molecular weight substances had decreased below 1% of the signal from maleimide-albumin.

The measurements of the light absorption of the original and the processed albumin solution showed that each albumin molecule had been functionalized with 16.3 maleimide groups by the membrane reactor process. The yield was 57% of the original albumin used (see Table 1 below).

TABLE 1

Light absorption

| | (Diluted 1:10 in PBS) | | Concentration ¹ | | Loading | Volumes | Yield |
|---|---|---|---|---|---|---|---|
| | 280 nm | 310 nm | Albumin | Maleimide | Maleimide | | |
| | | | (mmol/l) | (mmol/l) | per Albumin | | |
| Original | 0.582 | 0.042 | 0.15 | 0.07 | <1 | 200 ml | 100% |
| Albumin | | | | | | | |
| solution | | | | | | | |
| Processed | 0.860 | 0.192 | 0.19 | 3.1 | 16.3 | 90 ml | 57% |
| Albumin | | | | | | | |
| solution | | | | | | | |

To detect gel formation, maleimide-albumin was initially concentrated 5-fold by means of freeze-drying after processing in the membrane reactor. To test for gel formation, 3 μl of 10-fold buffer (pH 7.2), 7 μl of water, 10 μl of the concentrated albumin solution and 10 μl of bis(thio)polyethylene glycol (20 mmol/L thiol groups) were mixed with one another. A hydrogel was formed from this solution within 20 s after mixing.

### 3. CONCLUSION

The covalent coupling of maleimide groups to albumin in a membrane reactor was evaluated. A dialysis cassette, as is used to purify blood, was used as a membrane reactor. All the necessary steps in the coupling to functionalize, purify and concentrate were carried out using the reactor system in which the albumin solution remained in a closed loop throughout the entire process. In this process, 16.3 maleimide groups were coupled per albumin molecule. The albumin yield was 57% and the purity was more than 99%. The maleimide-albumin prepared in this manner is suitable for gel formation using suitable thiol-containing crosslinkers.

1. A method of preparing a product comprising:
passing a first liquid comprising component A along one side of a semi-permeable membrane, and
passing a second liquid comprising component B along another side of the membrane,
wherein the membrane excludes passage of component A but allows passage of component B and, after passage of component B through the membrane, a chemical reaction takes place between component A and component B.

2. The method according to claim 1, wherein the product is a functionalized high molecular weight component.

3. The method according to claim 1, wherein the product is a functionalized protein.

4. The method according to claim 1, wherein the another side is an opposing side of the membrane.

5. The method according to claim 1, wherein the chemical reaction between component A and component B takes place with formation of covalent bonds.

6. The method according to claim 1, wherein preparation of the product includes purification and/or concentration thereof.

7. The method according to claim 1, wherein the second liquid is guided along the membrane countercurrent to the first liquid.

8. The method according to claim 1, wherein the first and second liquid are each guided along the membrane via a closed loop.

9. The method according to claim 1, wherein preparation of the product is carried out in a dialyser containing the membrane.

10. The method according to claim 1, wherein preparation of the product is carried out by a dialysis device or a dialysis apparatus or equipment.

11. The method according to claim 1, wherein the second liquid, in addition to component B, comprises a membrane-permeable component C or, after the second liquid, a third liquid comprising a membrane-permeable component C is guided along the membrane.

12. The method according to claim 11, wherein, after passage of component C through the membrane, a chemical reaction also takes place between component A and component C.

13. The method according to claim 12, wherein the chemical reaction between component A and component C takes place with formation of covalent bonds.

14. The method according to claim 12, wherein component C reacts with other functional groups of component A than component B.

15. The method according to claim 1, wherein component A is selected from the group consisting of proteins, enzymes, antibodies, extracellular proteins, hormones, growth factors, cytokines, polysaccharides, salts thereof, derivatives thereof, conjugates thereof and mixtures thereof.

16. The method according to claim 1, wherein component A is albumin.

17. The method according to claim 1, wherein component A is serum albumin.

18. The method of claim 1, wherein component A is human serum albumin.

19. The method according to claim 1, wherein component B and/or an optional component C is a thiol-reactive compound or a maleimide derivative.

20. The method according to claim 19, wherein the thiol-reactive compound is selected from the group consisting of maleimide compounds, vinyl sulphone compounds, acrylate compounds, alkyl halide compounds, azirine compounds, pyridine compounds, thionitrobenzoic acid compounds, aryl compounds, derivates thereof and mixtures thereof.

21. The method according to claim 1, wherein component B and/or an optional component C are selected from the group consisting of 3-maleimidopropionic acid, 3-maleimidopropionic acid N-hydroxysuccinimide ester and combinations thereof.

22. A product prepared or which may be prepared by the method according to claim 1.

23. The product according to claim 22, which is a functionalized high molecular weight component.

24. The product according to claim 22, which is a functionalized protein.
